SEQUENCE LISTING (2 sequences)

SEQ ID NO: 1 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 14 aa, Synthetic construct
IEGPTLRQWLAARA

(19) United States
(12) Patent Application Publication
(10) Pub. No.: US 2026/0355281 A1
(43) Pub. Date: Nov. 12, 2026

(54) STABLE PHARMACEUTICAL COMPOSITIONS COMPRISING ROMIPLOSTIM

(71) Applicant: SYNA THERAPEUTICS, S.L., Barcelona (ES)

(72) Inventors: Natalia CAMPOS HEREDIA, Sant Joan Despí (ES); Gemma LAHUERTA BUIRA, Sant Joan Despí (ES); Andreu SOLDEVILA FABREGAS, Barcelona (ES); Carlos NIETO ABAD, Sant Joan Despí (ES)

(73) Assignee: SYNA THERAPEUTICS, S.L., Barcelona (ES)

(21) Appl. No.: 19/139,654

(22) PCT Filed: Dec. 22, 2023
(86) PCT No.: PCT/EP2023/087664

§ 371 Date: Jun. 16, 2025

(30) Foreign Application Priority Data
Dec. 23, 2022 (EP) 22383287.4

(51) Int. Cl. A61K 38/19; A61K 9/19; A61K 47/02; A61K 47/18; A61K 47/26

(52) U.S. Cl. CPC A61K 38/196; A61K 9/19; A61K 47/02; A61K 47/183; A61K 47/26

(57) ABSTRACT

A pharmaceutical composition comprising: (i) a therapeutically effective amount of romiplostim; and (ii) a combination of excipients comprising: —a pH buffering agent with a pH from 4.5 to 5.5; —mannitol at an amount from 4.5 to 5.5% w/v; —trehalose at an amount of 3% w/v; and—a surfactant at an amount from 0.003 to 0.010 w/v. A method for preparing said pharmaceutical composition by mixing the excipients with romiplostim. A method for preparing a lyophilized form of said pharmaceutical composition and a method and a kit for preparing its reconstituted form. Said compositions for use in the treatment or prevention of thrombocytopenia; particularly of idiopathic or immune thrombocytopenia (ITP).

### FIELD OF THE INVENTION

The present invention relates to the field of pharmaceutical compositions. Concretely, the present invention provides a particular combination of excipients which is able to confer stability to an active ingredient, as well as pharmaceutical compositions, particularly lyophilized pharmaceutical compositions, comprising thereof and processes for their preparation.

### BACKGROUND OF THE INVENTION

Generally, proteins have a very short half-life, and undergo denaturation (such as aggregation, dissociation, and adsorption on the surface of vessels) upon exposure to various factors such as unfavorable temperatures, water-air interface, high-pressure, physical/mechanical stress, organic solvents and microbial contamination. Consequently, the denatured protein loses intrinsic physicochemical properties and physiological activity. Denaturation of proteins is often irreversible, and therefore proteins, once denatured, may not recover their native properties to the initial state.

To overcome the stability problem of proteins in aqueous formulations, therapeutic protein products are made more stable via lyophilization (freeze-drying). Lyophilized products are usually accompanied by sterile aqueous media for reconstitution. After reconstitution, the formulations typically have short useful storage lives, even when stored at low temperatures (e.g., 5 °C)

Pharmaceutical formulations that must be refrigerated have several drawbacks. For example, the formulations must be stored at refrigerated temperatures in order to achieve a commercially viable shelf-life from a drug product supply chain perspective. Refrigeration of drug product also impacts the cost of goods. Furthermore, refrigerated products are inconvenient because they require special handling procedures prior to administration.

Therefore, there is still the need of improving the stability of lyophilized pharmaceutical compositions.

### SUMMARY OF THE INVENTION

The inventors of the present invention have developed a particular combination of excipients which, when formulated together with the active ingredient into a lyophilized composition, confers a remarkable stability at room temperature both to the active ingredient as well as to the lyophilized formulation as such

Thus, in a first aspect the present invention provides a combination of excipients comprising: a pH buffering agent with a pH from 4.5 to 5.5% w/v; trehalose at an amount of 3% w/v; and a surfactant at an amount from 0.003 to 0.010 w/v.

As it is shown below, the inventors prepared a lyophilized peptibody composition comprising the above-mentioned combination of excipients together with romiplostim. And performed several tests to evaluate its behaviour in front of the composition currently marketed under the trademark Nplate®, which includes the same active ingredient, at the same amount, but with a different excipient composition.

The main findings (summarized in Table 3 below) were:
I. Cake appearance: at room temperature (25 °C), after 6 months of storage, the cake appearance of the comparative formulation was altered in the 25% of the vials; contrarily to the formulation of the invention, which maintained the 100% of the vials classified as Class I.
II. Aggregate formation: at room temperature, the comparative formulation included 0.06% of aggregates. On the contrary, the formulation of the invention, including the particular excipient combination, reduced to 0% the amount of aggregates.
This stability against aggregate formation was further confirmed when the test was performed under stress conditions of temperature (37 °C), wherein it was found a remarkable difference: in the case of the comparative composition a 0.84% of aggregates were produced, whereas in the case of the invention the amount was of 0.04%.
III. It was found that under storage refrigerator T, the comparative formulation significantly lost some active ingredient with time (showing about 93% after 12 months), contrary to the formulation of the invention which, after 12 months, retained the 100% of the active ingredient.
IV. Formation of basic species: under stress T conditions (37 °C) it was found that the comparative formulation had, after 3 months, a significant higher amount of basic specie.

Altogether, the present findings allow to conclude that the particular excipient combination of the present invention, including mannitol in an amount within the range from 4.5 to 5.5% w/v, and trehalose at a 3% w/v, stabilizes the lyophilized composition in front of temperature and time.

This means a great advance in the field of pharmaceutical formulations, particularly of lyophilized pharmaceutical compositions, wherein one of the main limitations is the need of refrigeration in order to guarantee the maximum stability and bioavailability of the active ingredient with time.

In a second aspect the present invention provides a pharmaceutical composition, particularly a lyophilized pharmaceutical composition, comprising a therapeutically effective amount of an active ingredient together with the combination of excipients as defined in the first aspect of the invention.

In a third aspect the present invention provides a method for preparing a pharmaceutical composition as defined in the second aspect of the invention, the method comprising the step of mixing the active ingredient and the excipients forming part of the combination as defined in the first aspect of the invention.

In a fourth aspect the present invention provides a method for preparing a lyophilized pharmaceutical composition as defined in the second aspect of the invention, the method comprising the steps of (i) preparing a solution comprising the combination of excipients as defined in the first aspect of the invention, and the active ingredient; and (ii) lyophilizing the resulting solution.

In a fifth aspect the present invention provides a method for preparing a reconstituted pharmaceutical composition comprising the step of reconstituting the lyophilized composition as defined in the fourth aspect of the invention.

In a sixth aspect the present invention provides a kit for preparing an aqueous pharmaceutical composition having a lyophilized pharmaceutical composition as defined in the fifth aspect of the invention and a second physiologically acceptable solvent for reconstituting the composition.

In a final aspect the present invention provides the pharmaceutical composition or kit as provided by the present invention for use in the treatment or prevention of thrombocytopenia; particularly for treating or preventing idiopathic or immune thrombocytopenia (ITP). This aspect can alternatively be formulated as the use of a pharmaceutical composition for use in the manufacture of a medicament for the treatment or prevention of thrombocytopenia; particularly for treating or preventing idiopathic or immune thrombocytopenia (ITP). This aspect can alternatively be formulated as a method for the treatment or prevention of thrombocytopenia; particularly for treating or preventing idiopathic or immune thrombocytopenia (ITP), the method comprising administering the pharmaceutical composition of the invention to a subject in need thereof or using the kit of the invention.

### DETAILED DESCRIPTION OF THE INVENTION

Terms not specifically defined herein should be given the meanings that would be given to them by one of skill in the art in light of the disclosure and the context. As used in the specification, however, unless specified to the contrary, the following terms have the meaning indicated and the following conventions are adhered to.

Throughout the present specification and the accompanying clauses, the words “comprise” and variations such as “comprises”, “comprising” are to be interpreted inclusively. That is, these words are intended to convey the possible inclusion of other elements or integers not specifically recited, where the context allows. The word “comprise” also includes the term “consists of”.

For the purposes of the present invention, any ranges given include both the lower and the upper end-points of the range.

In a first aspect the present invention provides a combination of excipients as defined in the first aspect of the invention.

In the context of the present invention, the expression “% w/v” means the percentage weight (mg) of a single ingredient relative to the total volume of the entire formulation (either the combination or pharmaceutical composition).

In one embodiment of the first aspect of the invention, optionally in combination with any of the embodiments provided below, the mannitol is at a 4.5%, 4.6%, 4.7%, 4.8%, 4.9%, 5.0%, 5.1%, 5.2%, 5.3%, 5.4% or 5.5% w/v. In a particular embodiment, optionally in combination with any of the embodiments provided below, the mannitol is at a 5.0%.

In one embodiment of the first aspect of the invention, optionally in combination with any of the embodiments provided above or below, the surfactant is at an amount from 0.003 to 0.008% w/v, particularly in an amount of 0.003, 0.004, 0.005, 0.006, 0.007, or 0.008% w/v. In a particular embodiment, optionally in combination with any of the embodiments provided above or below, the surfactant is at an amount of 0.004% w/v.

As used herein, “buffering agent” refers to a buffered solution that resists changes in pH by the action of its acid-base conjugate components. Buffering agent is used in the present invention to maintain the pH from 4.5 to 5.5, more particularly at pH 4.5, 4.6, 4.7, 4.8, 4.9, 5.0, 5.1, 5.2, 5.3, 5.4, or 5.5. In one embodiment of the first aspect of the invention, optionally in combination with any of the embodiments provided above or below, the pH is 5.0.

In one embodiment of the first aspect of the invention, optionally in combination with any of the embodiments provided above or below, the buffering agent is selected from the group consisting of glycine, histidine, glutamate, succinate, phosphate, acetate, and aspartate; particularly, the buffering agent is histidine.

In one embodiment of the first aspect of the invention, optionally in combination with any of the embodiments provided above or below, the buffering agent is histidine and the pH is from 4.5 to 5.5, particularly at a pH 5.

In one embodiment of the first aspect of the invention, optionally in combination with any of the embodiments provided above or below, the buffering agent is at a concentration from 1 mM to 100 mM, particularly from 5 mM to 25 mM.

In another embodiment of the first aspect of the invention, optionally in combination with any of the embodiments provided above or below, the buffering agent is histidine, at a concentration from 5 to 25 mM and a pH from 4.5 to 5.5. In another embodiment of the first aspect of the invention, optionally in combination with any of the embodiments provided above or below, the buffering agent is histidine, at a concentration from 5 to 25, particularly of 10 mM, and a pH of 5.

In the context of the invention, the surfactant is used in order to prevent adsorption of the active ingredient on the surface of the vial, ampoule, carpoule, cartridge or syringe. Surfactants lower surface tension of a protein solution, thereby, preventing its adsorption or aggregation on to a hydrophobic surface.

In one embodiment of the first aspect of the invention, optionally in combination with any of the embodiments provided above or below, the surfactant is selected from the group consisting of sodium lauryl sulfate, dioctyl sodium sulfosuccinate, dioctyl sodium sulfonate, poloxamer, chenodeoxycholic acid, N-lauroylsarcosine sodium salt, lithium dodecyl sulfate, 1-octanesulfonic acid sodium salt, sodium cholate hydrate, sodium deoxycholate, glycodeoxycholic acid sodium salt, benzalkonium chloride or 30 benzethonium chloride, cetylpyridinium chloride monohydrate, hexadecyltrimethylammonium bromide, 3-((3-cholamidopropyl)dimethylammonio)-1-propanesulfonate (CHAPS), 3-([3-Cholamidopropyl]dimethylammonio)-2-hydroxy-1-propanesulfonate (CHAPSO), SB3-10, SB3-12, digitonin, Triton X-100, Triton X-114, lauromacrogol 400, polyoxyl 40 stearate, polyoxyethylene hydrogenated castor oil 10, 40, 50 and 60, glycerol monostearate, polysorbate-based surfactant (such as polysorbate 20, 40, 60, 65 and 80), poloxamer-based surfactant, soy lecithin, Dipalmitoylphosphatidylcholine (DOPC), 1,2-Dimyristoyl-sn-glycero-3-phosphorylglycerol (DMPG), Dipalmitoylphosphatidylcholine (DMPC), and 18:1/18:1-Dioleoyl-phosphatidylgycerol (DOPG); sucrose fatty acid ester, methyl cellulose and carboxymethyl cellulose; particularly, the surfactant is selected from a polysorbate-based non-ionic surfactant, a poloxamer-based non-ionic surfactant, or a combination thereof. Particularly, the surfactant is a polysorbate-based non-ionic surfactant; more particularly it is polysorbate 20.

In one embodiment of the first aspect of the invention, optionally in combination with any of the embodiments provided above or below, the surfactant is present at a concentration from 0.001% w/v to 0.008% w/v.

In one embodiment of the first aspect of the invention, optionally in combination with any of the embodiments provided above or below, the combination of excipients is selected from the following group:
I) histidine from 1 to 100 mM, pH 5; mannitol at 5% w/v; trehalose at 3% w/v; and polysorbate or poloxamer from 0.001 to 0.010% w/V;
II) histidine from 5 to 25 mM, pH 5; mannitol at 5% w/v; trehalose at 3% w/v; and polysorbate or poloxamer from 0.004 to 0.008% w/v;
III) histidine 10 mM, pH 5; mannitol at 5% w/v; trehalose at 3% w/v; and polysorbate or poloxamer at 0.004% w/v; or
IV) histidine 10 mM, pH 5; mannitol at 5% w/v; trehalose at 3% w/v; and polysorbate, particularly polysorbate 20, at 0.004% w/v.

In a further aspect, the invention provides a pharmaceutical composition, particularly a lyophilized pharmaceutical composition, comprising a therapeutically effective amount of an active ingredient together with the combination of excipients as defined in any of the preceding claims.

By “therapeutically effective amount”, it is understood the amount of the active ingredient that, when administered, is sufficient to prevent development of, or alleviate to some extent, one or more of the symptoms of the disease which is addressed.

The precise therapeutic dose of the component(s), may depend on several variables. Some of these would be: route of administration, time of drug release (e.g., instant or extended), administration schedule, pain severity, condition of the patient, and the like.

In one embodiment of the invention, optionally in combination with any of the embodiments provided above or below, the pharmaceutical composition, and particularly the lyophilized pharmaceutical composition, comprises an active ingredient which is selected from a polypeptide that binds to a protein; an aptamer; or a nucleic acid molecule that binds to a nucleic acid molecule encoding a target (e.g., an siRNA).

In one embodiment of the invention, optionally in combination with any of the embodiments provided above or below, the pharmaceutical composition, and particularly the lyophilized pharmaceutical composition, comprises an active ingredient which is a polypeptide that binds to a protein. Illustrative non-limitative examples of polypeptides that binds to a protein are antibodies, immunoadhesins, or peptibodies. In one particular embodiment, optionally in combination with any of the embodiments provided above or below, the pharmaceutical composition comprises a peptibody as active ingredient.

In the context of the invention, a “peptibody” (also known as “Fc-peptide fusion protein”) refers to a molecule comprising peptide(s) fused either directly or indirectly to other molecules such as an Fc domain of an antibody, where the peptide moiety specifically binds to a desired target. The peptide(s) may be fused to either an Fc region or inserted into an Fc-Loop, a modified Fc molecule. Fc-Loops are described in U.S. Patent Application Publication No. US2006/0140934, whose content is also included herein by reference.

In one embodiment of the invention, optionally in combination with any of the embodiments provided above or below, the peptibody comprises structure of formula (I):

[(X¹)ₐ—F¹—(X²)_{b}]-(L¹)_{c}-WSP_{d}
wherein
X¹ is selected from:
P¹-(L²)e-
P²-(L³)f-P¹-(L²)e-
P³-(L⁴)g-P²-(L³)f-P¹-(L²)e- and
P⁴-(L⁵)h-P³-(L⁴)g P²-(L³)f-P¹-(L²)e;
F¹ is a Fc domain;
X² is selected from:
   (L²)e-P¹
   (L²)e-P¹-(L³)f-P²
   (L²)e-P¹-(L³)f-P²-(L⁴)g-P³, and
   (L²)e-P¹-(L³)f-P²-(L⁴)f-P³-(L⁵)h-P⁴;
wherein
P¹, P²; P³, and P⁴ are each independently sequences of pharmacologically active peptides;
L¹, L², L³, L⁴, and L⁵ are each independently linkers; a, b, c, e, f, g, and h are each independently 0 or 1, provided that at least one of a and b is 1; d is 0 or 1; and
WSP is a water soluble polymer, the attachment of which is effected at any reactive moiety in F¹.

In one embodiment of the invention, optionally in combination with any of the embodiments provided above or below, the peptibody comprises formula (II):

[F¹-(L¹)ₑ-P¹-(L²)_{f}-P²]-(L¹)_{c} (II)

In one embodiment of the invention, optionally in combination with any of the embodiments provided above or below, the peptibody is a multimer or dimer; particularly a dimer.

In one embodiment of the invention, optionally in combination with any of the embodiments provided above or below, the peptibody is one wherein P¹, P² and P³ have the same amino acid sequence.

In one embodiment of the invention, optionally in combination with any of the embodiments provided above or below, the peptibody is one wherein F¹ is of sequence SEQ ID NO: 1.

In one embodiment of the invention, optionally in combination with any of the embodiments provided above or below, the peptibody is one wherein wherein L¹ and L² are polyglycine peptides. In one embodiment of the invention, optionally in combination with any of the embodiments provided above or below, L¹ is (Gly)₅. In another embodiment, optionally in combination with any of the embodiments provided above or below, L² is (Gly)₈. In another embodiment of the invention, optionally in combination with any of the embodiments provided above or below, L¹ is (Gly)₅ and L² is (Gly)₈.

P¹, P², P³ and/or P⁴ peptides are independently selected from a peptide provided in any of the Tables 4 to 38 of EP2018183, whose content is incorporated herein by reference.

In one embodiment of the invention, optionally in combination with any of the embodiments provided above or below, P¹ and P² are of sequence SEQ ID NO: 2.

In another embodiment of the invention, optionally in combination with any of the embodiments provided above or below, Fc is of sequence SEQ ID NO: 1, P¹ and P² are of sequence SEQ ID NO: 2, L¹ is (Gly)₅, L² is (Gly)₈, e=f=1 and c=d=0.

In another embodiment of the invention, optionally in combination with any of the embodiments provided above or below, the peptibody is romiplostim (CAS number 267639-76-9).

In another embodiment of the invention, optionally in combination with any of the embodiments provided above or below, the peptibody is at a concentration between 0.25 and 250 mg/mL, particularly at a concentration of 0.5 mg/mL.

In a further embodiment, optionally in combination with any of the embodiments provided above or below, the invention provides a pharmaceutical composition which is selected from the group consisting of:
I) 0.5 mg/mL of romiplostim; histidine from 1 to 100 mM, pH 5; mannitol at 5% w/v; trehalose at 3% w/v; and polysorbate or poloxamer from 0.001 to 0.010% w/v;
II) 0.5 mg/mL of romiplostim; histidine from 5 to 25 mM, pH 5; mannitol at 5% w/v; trehalose at 3% w/v; and polysorbate or poloxamer from 0.004 to 0.008% w/v;
III) 0.5 mg/ml of romiplostim; histidine 10 mM, pH 5; mannitol at 5% w/v; trehalose at 3% w/v; and polysorbate or poloxamer at 0.004% w/v; and
IV) 0.5 mg/ml of romiplostim; histidine 10 mM, pH 5.0; mannitol 5% w/v; trehalose 3% w/v; and polysorbate, particularly polysorbate 20, 0.004% w/v.

In a further embodiment, optionally in combination with any of the embodiments provided above or below, the invention provides a lyophilized pharmaceutical composition which is selected from the group consisting of:
I) 0.5 mg/ml of romiplostim; histidine from 1 to 100 mM, pH 5; mannitol at 5% w/v; trehalose at 3% w/v; and polysorbate or poloxamer from 0.001 to 0.010% w/v;
II) 0.5 mg/mL of romiplostim; histidine from 5 to 25 mM, pH 5; mannitol at 5% w/v; trehalose at 3% w/v; and polysorbate or poloxamer from 0.004 to 0.008% w/v;
III) 0.5 mg/ml of romiplostim; histidine 10 mM, pH 5; mannitol at 5% w/v; trehalose at 3% w/v; and polysorbate or poloxamer at 0.004% w/v; and
IV) 0.5 mg/mL of romiplostim; histidine 10 mM, pH 5.0; mannitol 5% w/v; trehalose 3% w/v; and polysorbate, particularly polysorbate 20, 0.004% w/v.

Particularly, the pharmaceutical composition of the second aspect of the invention is a lyophilized composition comprising 0.5 mg/ml of romiplostim; histidine 10 mM, pH 5.0; mannitol 5% w/v; trehalose 3% w/v; and polysorbate, particularly polysorbate 20, 0.004% w/v.

In a further aspect the present invention provides a method for preparing a pharmaceutical composition of the invention. The method comprises mixing the different excipients and the active ingredient, in any order.

There are routine means to perform the mixing step. No particular limitations exist.

In a further aspect the present invention provides a method for preparing a lyophilized pharmaceutical composition of the invention, the method comprising the steps of (i) preparing a solution comprising the combination of excipients as defined in any of the above aspects and embodiments; and (ii) lyophilizing the resulting solution.

Lyophilization is carried out using techniques common in the art and consists of a water removal process typically used to preserve perishable materials, to extend shelf life or make the material more convenient for transport. Lyophilization works by freezing the material, then reducing the pressure and adding heat to allow the frozen water in the material to sublimate.

There are various methods to freezing the product. Freezing can be done in a freezer, a chilled bath (shell freezer) or on a shelf in the freeze dryer. Cooling the material below its triple point ensures that sublimation, rather than melting, will occur. This preserves its physical form.

Lyophilization's second phase is primary drying (sublimation), in which the pressure is lowered and heat is added to the material in order for the water to sublimate. The vacuum speeds sublimation. The cold condenser provides a surface for the water vapor to adhere and solidify. The condenser also protects the vacuum pump from the water vapor. About 95% of the water in the material is removed in this phase.

Lyophilization's final phase is secondary drying (adsorption), during which the ionically-bound water molecules are removed. By raising the temperature higher than in the primary drying phase, the bonds are broken between the material and the water molecules. Freeze dried materials retain a porous structure. After the lyophilization process is complete, the vacuum can be broken with an inert gas before the material is sealed. Most materials can be dried to 1-5% residual moisture.

In a further aspect the present invention provides a method for preparing a reconstituted pharmaceutical composition comprising the step of reconstituting the lyophilized composition as defined in any of the above embodiments.

The standard reconstitution practice for lyophilized material is to add back a volume of pure water or sterile water for injection (WFI) (typically equivalent to the volume removed during lyophilization), although dilute solutions of antibacterial agents are sometimes used in the production of pharmaceuticals for parenteral administration.

The lyophilized therapeutic composition may be reconstituted as an aqueous solution. A variety of aqueous carriers, e.g., sterile water for injection, water with preservatives for multi dose use, or water with appropriate amounts of surfactants (for example, polysorbate-20), 0.4% saline, 0.3% glycine, or aqueous suspensions may contain the active compound in admixture with excipients suitable for the manufacture of aqueous suspensions. In various embodiments, such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The therapeutic compositions may be administered orally, topically, transdermally, parenterally, by inhalation spray, vaginally, rectally, or by intracranial injection. The term parenteral as used herein includes subcutaneous injections, intravenous, intramuscular, intracisternal injection, or infusion.techniques. Administration by intravenous, intradermal, intramusclar, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient.

Single or multiple administrations of the compositions can be carried out with the dose levels and pattern being selected by the treating physician. For the prevention or treatment of disease, the appropriate dosage will depend on the type of disease to be treated, as defined above, the severity and course of the disease, whether drug is administered for preventive or therapeutic purposes, previous therapy, the patient's clinical history and response to the drug, and the discretion of the attending physician.

In a further aspect the present invention provides a kit for preparing an aqueous pharmaceutical composition having a lyophilized pharmaceutical composition as defined in any of the above embodiments and a second physiologically acceptable solvent for reconstituting the composition.

In one embodiment of the invention, optionally in combination with any of the embodiments provided above or below, the kit includes the lyophilized composition as defined in the third aspect and any embodiment thereof, packaged in a container such as a sealed bottle or vessel, with a label affixed to the container or included in the package that describes use of the compound or composition in practicing the method. In a further embodiment, optionally in combination with any of the embodiments provided above or below, the kit contains a first container having the lyophilized composition of the invention, as defined in the third aspect of the invention of any embodiment thereof, and a second container having a physiologically acceptable reconstitution solution for the lyophilized composition. In one embodiment of the invention, optionally in combination with any of the embodiments provided above or below, the lyophilized composition is packaged in a unit dosage form. The kit may further include a device suitable for administering the composition according to a specific route of administration. In a particular embodiment, the kit further contains a label that describes use of the therapeutic protein or peptide composition. In one embodiment, optionally in combination with any of the embodiments provided above, the kit is for preparing an aqueous pharmaceutical composition including romiplostim as active ingredient.

Both the pharmaceutical composition and the kit of the present invention, when include romiplostim as active ingredient, can be used with any of the therapeutic indications already approved by the Medicine Agencies such as EMA or FDA. Therefore, in a final aspect the present invention provides the pharmaceutical composition or kit as defined above, which includes as active ingredient romiplostim, for use in the treatment or prevention of thrombocytopenia; particularly for treating or preventing idiopathic or immune thrombocytopenia (ITP).

Throughout the present description, the term “treatment” includes, but is not limited to, alleviating, diminishing or eliminating one or more symptoms of the disease; reducing the degree of the disease, stabilizing (i.e., not worsening) the condition, delaying or slowing the progression of the disease, alleviating or improving its condition, and remitting (whether total or partial).

As used in the present invention, the term “prevention” refers to preventing the onset of the disease from occurring in a patient who is predisposed, but who does not yet have symptoms of the disease.

As used herein, the term “subject” shall refer to a vertebrate animal. Such animals include both domestic animals; for example, livestock, laboratory animals and household pets, and non-domestic animals such as wildlife. In one embodiment, the animal is a vertebrate. In a particular embodiment the animal is a domestic mammal or a human.

To those skilled in the art, other objects, advantages or features of the invention will be apparent in part from the description or in part from the practice of the invention. The following examples are provided by way of illustration or are not intended to be limiting of the present invention.

### Examples

### Example 1: Preparation of the Lyophilised Romiplostim Formulations

The formulations of Table 1 (invention) and 2 (comparative purpose) were prepared:

TABLE 1
Formulation of the invention

| | CAS | | | | |
|---|---|---|---|---|---|
| Component | number | Supplier | Function | Quantity | |
| Romiplostim | 267639- | Internal | TPO receptor | 0.5 | mg/mL |
| | 76-9 | | agonist | | |
| D-mannitol | 87-78-5 | Pfanstiehl | Bulking agent | 5% | w/v |
| Trehalose | 6138- | Glentham | Stabilizer | 3% | w/v |
| | 23-4 | Life | | | |
| | | Sciende | | | |
| L-histidine | 71-00-1 | Merck | Buffering agent | 10 | mM |
| Polysorbate | 9005- | Croda | Stabilizer | 0.004% | w/v |
| 20 | 64-5 | | (prevention of | | |
| | | | aggregation) | | |
| Dillute | 7647- | | pH adjustment | | |
| Hydrochlo- | 01-0 | | (to target | | |
| ride acid | | | pH of 5.0) | | |

TABLE 2
Formulation for comparative purposes

| | CAS | | | | |
|---|---|---|---|---|---|
| Component | number | Supplier | Function | Quantity | |
| Romiplostim | 267639- | Internal | TPO receptor | 0.5 | mg/mL |
| | 76-9 | | agonist | | |
| D-mannitol | 87-78-5 | Pfanstiehl | Bulking agent | 4% | w/v |
| Sucrose | 57-50-1 | Merck | Stabilizer | 2% | w/v |
| L-histidine | 71-00-1 | Merck | Buffering agent | 10 | mM |
| Polysorbate | 9005- | Croda | Stabilizer | 0.004% | w/v |
| 20 | 64-5 | | (prevention of | | |
| | | | aggregation) | | |
| Dillute | 7647- | | pH adjustment | | |
| Hydrochlo- | 01-0 | | (to target | | |
| ride acid | | | pH of 5.0) | | |

Romiplostim was synthesized as disclosed in Grant Shimamoto G. et al., “Peptibodies-a flexible alternative format to antibodies”, mAbs, 4(5), 586-591; September/October 2012; 2012.

Next, Romiplostim was thawed and formulated to 0.5 mg/ml by the addition of the excipients pointed out in each of the above tables. Each one of the resulting formulations was 0.22 μm filtered into a sterilised vessel.

The solution was sterilized by filtration with 0.22 μm-pore sized filters, using a peristaltic pump to generate filtration driving force. Then, it was filled into vials (1.25 ml/vial) and it was subsequently lyophilized. Lyophilization (equipment Telstar Lyobeta 20) consisted of a freezing step first (T<−30 °C, for >3 h), followed by a sublimation step (P<1 mbar, T<−10 °C until end point) and a water-desorption step (T>10 °C, vacuum). Finally, the vials were stoppered and capped.

### Example 2: Packing of the Lyophilized Formulation

a) Primary packing (formulation C-comparative purpose)
   Type I Glass vials, 8 mL, mouth 20 mm
   Chlorobutyl rubber stopper, grey, mouth 20 mm
   Aluminium capsules
b) Primary packing (formulation I-formulation of the invention)
   Type I Glass vials, 8 mL, mouth 20 mm
   Teflonated stoppers, grey, mouth 20 mm
   Aluminium capsules

### Example 3: Comparative Tests of Stability

The stability of both formulations was assessed at refrigeration temperature, as well as at room temperature (25 °C) and stress temperature (37 °C). The following parameters were determined:
(a) Aggregate formation: size-exclusion HPLC (SEC-HPLC). SEC-HPLC analysis was performed, using a TSK gel UP-SW3000 4.6×300 mm column maintained at a temperature of 40 °C. The formulation (containing 5 mcg of protein) was eluted in isocratic mode using a potassium phosphate with potassium chloride and propanol mobile phase at a flow rate of 0.25 ml/min with UV detection at 215 nm
(b) Formation of basic species. Capillary isoelectic focusing electrophoresis was performed using a neutral coated capillary 50 μm I.D×30.2 cm in a PA800 plus equipment. Samples were concentrated up to 2 mg/ml and mixed with a solution containing 3.75M urea-cIEF fel, pharmalyte 3-10 (ampholytes), L-arginine, iminodiacetic acid and pl markers. The sample mixture was injected by pressure (15 pSi for 150 sec) into the cartridge and focused by applying high voltage (separation: 30 kV for 30 min) with detection at 280 nm.
(c) Cake appearance: the appearance of the cake was examinated by visual inspection in a total set of 23 samples of the formulation of the invention and 27 of the commercial formulation after storaging for 6 months at accelerated conditions and 3 months at stress conditions. The evaluation was performed considering as stable the one with no significant changes in lyophilized appearance (i.e., color or texture) compared to time zero. The results were expressed as the % of samples which resulted to be stable vs the total number of samples analysed.

### Results

The results are summarized in Table 3 below:

TABLE 3

| | | | Room temperature (25 °C) | | | Stress (37 °C) | |
|---|---|---|---|---|---|---|---|
| Quality attribute | Form. | T0 | T1 M | T3 M | T6 M | T1 M | T3 M |
| Cake appearance ⁽¹⁾ | C | 100% | ND | 100% | 75% | ND | 60% |
| | I | 100% | 100% | 100% | 100% | ND | 80% |
| HMWF by SEC-HPLC | C | ND | ND | 0.04 | 0.06 | ND | 0.84 |
| | I | 0 | 0 | 0 | 0 | 0 | 0.04 |
| Total Basic Variants | C | ND | ND | ND | ND | ND | 7.2 |
| (TBV) (%) | I | ND | ND | ND | ND | ND | 2.6 |

⁽¹⁾ determined as (the number of vials which remain intact with respect to the total number of vials) × 100

Both the composition of the invention, including the particular combination of excipients and the comparative one (Nplate®), were found to be stable under refrigeration.

However, when both formulations were subjected to room temperature conditions, the following was found:
1. After 6 months of storage, the cake appearance of the comparative formulation was altered in the 25% of the vials; contrarily to the formulation of the invention, which maintained the 100% of the vials classified as Class I.
2. At room temperature, the comparative formulation included 0.06% of aggregates after 6 month of storage. On the contrary, the formulation of the invention, including the particular excipient combination, was able completely efficient in avoiding formation of aggregates.
3. At refrigeration T, the comparative formulation significantly lost some active ingredient with time (showing about 93% after 12 months), contrary to the formulation of the invention which, after 12 months, retained the 100% of the active ingredient.

The above conclusions about the significant “protective” role of the combination of excipients in front of temperature, was further confirmed when the formulations were subjected to stress temperature (37 °C), especially in the formation of aggregates (in the case of the comparative composition a 0.84% was produced, whereas in the case of the invention the amount was of 0.04%), and in the formation of basic species.

Altogether the above data support that the pharmaceutical compositions comprising the combination of the invention are so stable at room temperature, for long periods of storage, that the active ingredient and formulation are in optimal condition for its use.

### CLAUSES

For reasons of completeness, various aspects of the invention are set out in the following numbered clauses:

Clause 1. A combination of excipients comprising:
a pH buffering agent with a pH from 4.5 to 5.5;
mannitol at an amount from 4.5 to 5.5% w/V;
trehalose at an amount of 3% w/v; and
a surfactant at an amount from 0.003 to 0.010 w/v.

Clause 2. The combination of clause 1, wherein the mannitol is at a 4.5%, 4.6%, 4.7%, 4.8%, 4.9%, 5.0%, 5.1%, 5.2%, 5.3%, 5.4% or 5.5% w/v, particular at a 5% w/v.

Clause 3. The combination of any one of the preceding clauses, wherein the surfactant is at an amount from 0.003 to 0.008; particularly in an amount of 0.003, 0.004, 0.005, 0.006, 0.007, or 0.008% w/v; more particularly in an amount of 0.004% w/v.

Clause 4. The combination of any one of the preceding clauses, wherein the pH buffering agent provides a pH is of 4.5, 4.6, 4.7, 4.8, 4.9, 5.0, 5.1, 5.2, 5.3, 5.4, or 5.5; more particularly, pH is 5.0.

Clause 5. The combination of any one of the preceding clauses, wherein the buffering agent is selected from the group consisting of glycine, histidine, glutamate, succinate, phosphate, acetate, and aspartate; particularly, the buffering agent is histidine.

Clause 6. The combination of any one of the preceding clauses, wherein the surfactant is selected from the group consisting of sodium lauryl sulfate, dioctyl sodium sulfosuccinate, dioctyl sodium sulfonate, poloxamer, chenodeoxycholic acid, N-lauroylsarcosine sodium salt, lithium dodecyl sulfate, 1-octanesulfonic acid sodium salt, sodium cholate hydrate, sodium deoxycholate, glycodeoxycholic acid sodium salt, benzalkonium chloride or benzethonium chloride, cetylpyridinium chloride monohydrate, hexadecyltrimethylammonium bromide, 3-((3-cholamidopropyl)dimethylammonio)-1-propanesulfonate (CHAPS), 3-([3-Cholamidopropyl]dimethylammonio)-2-hydroxy-1-propanesulfonate (CHAPSO), SB3-10, SB3-12, digitonin, Triton X-100, Triton X-114, lauromacrogol 400, polyoxyl 40 stearate, polyoxyethylene hydrogenated castor oil 10, 40, 50 and 60, glycerol monostearate, polysorbate 20, 40, 60, 65 and 80, soy lecithin, Dipalmitoylphosphatidylcholine (DOPC), 1,2-Dimyristoyl-sn-glycero-3-phosphorylglycerol (DMPG), Dipalmitoylphosphatidylcholine (DMPC), and 18:1/18:1-Dioleoyl-phosphatidylgycerol (DOPG); sucrose fatty acid ester, methyl cellulose and carboxymethyl cellulose; particularly, the surfactant is polysorbate 20, polysorbate 80, and poloxamer 188; more particularly it is polysorbate 20.

Clause 7. The combination of any one of the preceding clauses, which is selected from the group consisting of:
I) histidine from 1 to 100 mM, pH 5; mannitol at 5% w/v; trehalose at 3% w/v; and polysorbate or poloxamer from 0.001 to 0.010% w/v;
II) histidine from 5 to 25 mM, pH 5; mannitol at 5% w/v; trehalose at 3% w/v; and polysorbate or poloxamer from 0.004 to 0.008% w/v;
III) histidine 10 mM, pH 5; mannitol at 5% w/v; trehalose at 3% w/v; and polysorbate or poloxamer at 0.004% w/v; and
IV) histidine 10 mM, pH 5; mannitol at 5% w/v; trehalose at 3% w/v; and polysorbate, particularly polysorbate 20, at 0.004% w/v.

Clause 8. A pharmaceutical composition comprising a therapeutically effective amount of an active ingredient together with the combination of excipients as defined in any of the preceding clauses.

Clause 9. The pharmaceutical composition of clause 8, which is a lyophilized pharmaceutical composition.

Clause 10. The pharmaceutical composition of any of the clauses 8-9, wherein the active ingredient is selected from a polypeptide that binds to a protein such as, an antibody, immunoadhesin, or peptibody; an aptamer; or a nucleic acid molecule that binds to a nucleic acid molecule encoding a target (e.g., an siRNA).

Clause 11. The pharmaceutical composition of clause 10, wherein the active ingredient is a polypeptide.

Clause 12. The pharmaceutical composition of any one of the clauses 8-11, wherein the active ingredient is a peptibody.

Clause 13. The pharmaceutical composition of clause 12, wherein the peptibody comprises structure of formula (I):

[(X¹)ₐ—F¹—(X²)_{b}]-(L¹)_{c}-WSP_{d}
wherein
X¹ is selected from:
P¹-(L²)e-
P²-(L³)f-P¹-(L²)e-
P³-(L⁴)g-P²-(L³)f-P¹-(L²)e- and
P⁴-(L⁵)h-P³-(L⁴)g P²-(L³)f-P¹-(L²)e;
F¹ is a Fc domain;
X² is selected from:
   (L²)e-P¹.
   (L²)e-P¹-(L³)f-P²
   (L²)e-P¹-(L³)f-P²-(L⁴)g-P³, and
   (L²)e-P¹-(L³)f-P²-(L⁴)f-P³-(L⁵)h-P⁴;
wherein
P¹, P²; P³, and P⁴ are each independently sequences of pharmacologically active peptides;
L¹, L², L³, L⁴, and L⁵ are each independently linkers;
a, b, c, e, f, g, and h are each independently 0 or 1, provided that at least one of a and b is 1; d is 0 or 1; and
WSP is a water soluble polymer, the attachment of which is effected at any reactive moiety in F¹.

Clause 14. The pharmaceutical composition of clause 13, wherein the peptibody comprises formula (II):

[F¹-(L¹)ₑ-P¹-(L²)_{f}-P²]-(L¹)_{c} (II)

Clause 15. The pharmaceutical composition of any one of the clauses 13-14, wherein the peptibody is a multimer or dimer.

Clause 16. The pharmaceutical composition of any one of the clauses 13-15, wherein P¹, P² and P³ have the same amino acid sequence.

Clause 17. The pharmaceutical composition of any one of the clauses 13-16, wherein F¹ is of sequence SEQ ID NO: 1.

Clause 18. The pharmaceutical composition of any one of the clauses 13-17, wherein L¹ and L² are polyglycine peptides.

Clause 19. The pharmaceutical composition of clause 18, wherein L¹ is (Gly)₅.

Clause 20. The pharmaceutical composition of clause 18, wherein L² is (Gly)₈.

Clause 21. The pharmaceutical composition of any one of the clauses 13-20, wherein P¹ and P² are of sequence SEQ ID NO: 2.

Clause 22. The pharmaceutical composition of any one of the clauses 13-21, wherein Fc is of sequence SEQ ID NO: 1, P¹ and P² are of sequence SEQ ID NO: 2, L¹ is (Gly)₅, L² is (Gly)₈, e=f=1 and c=d=0.

Clause 23. The pharmaceutical composition of any one of the clauses 13-22, wherein the peptibody is a dimer comprising the structure as defined in clause 22 (romiplostim).

Clause 24. The pharmaceutical composition of any one of the preceding clauses, wherein the peptibody is at a concentration between 0.25 and 250 mg/mL.

Clause 25. A pharmaceutical composition selected from the group of compositions consisting of:
I) 0.5 mg/ml of romiplostim; histidine from 1 to 100 mM, pH 5; mannitol at 5% w/v; trehalose at 3% w/v; and polysorbate or poloxamer from 0.001 to 0.010% w/v;
II) 0.5 mg/mL of romiplostim; histidine from 5 to 25 mM, pH 5; mannitol at 5% w/v; trehalose at 3% w/v; and polysorbate or poloxamer from 0.004 to 0.008% w/v;
III) 0.5 mg/ml of romiplostim; histidine 10 mM, pH 5; mannitol at 5% w/v; trehalose at 3% w/v; and polysorbate or poloxamer at 0.004% w/v; and
IV) 0.5 mg/mL of romiplostim; histidine 10 mM, pH 5.0; mannitol 5% w/v; trehalose 3% w/v; and polysorbate, particularly polysorbate 20, 0.004% w/v.

Clause 26. A lyophilized pharmaceutical composition selected from the group of compositions consisting of:
I) 0.5 mg/mL of romiplostim; histidine from 1 to 100 mM, pH 5; mannitol at 5% w/v; trehalose at 3% w/v; and polysorbate or poloxamer from 0.001 to 0.010% w/v;
II) 0.5 mg/ml of romiplostim; histidine from 5 to 25 mM, pH 5; mannitol at 5% w/v; trehalose at 3% w/v; and polysorbate or poloxamer from 0.004 to 0.008% w/v;
III) 0.5 mg/ml of romiplostim; histidine 10 mM, pH 5; mannitol at 5% w/v; trehalose at 3% w/v; and polysorbate or poloxamer at 0.004% w/v; and
IV) 0.5 mg/ml of romiplostim; histidine 10 mM, pH 5.0; mannitol 5% w/v; trehalose 3% w/v; and polysorbate, particularly polysorbate 20, 0.004% w/v.

Clause 27. A method for preparing a pharmaceutical composition as defined in any one of the clauses 8, 10-25, the method comprising the step of: mixing the combination of excipients as defined in any of the clauses 1-7 with the active ingredient.

Clause 28. A method for preparing a lyophilized pharmaceutical composition as defined in any one of the clauses 9-24 and 26, the method comprising the steps of (i) preparing a solution comprising the combination of excipients as defined in any of the preceding clauses 1-7 and the active ingredient; and (ii) lyophilizing the resulting solution.

Clause 29. A method for preparing a reconstituted pharmaceutical composition comprising the step of reconstituting the lyophilized composition as defined in any of the clauses 9-25.

Clause 30. A kit for preparing an aqueous pharmaceutical composition having a lyophilized pharmaceutical composition as defined in any of the clauses 9-24 and 26 and a second physiologically acceptable solvent for reconstituting the composition.

1. A pharmaceutical composition comprising:
(i) a therapeutically effective amount of romiplostim; and
(ii) a combination of excipients comprising:
a pH buffering agent with a pH from 4.5 to 5.5;
mannitol at an amount from 4.5 to 5.5% w/V;
trehalose at an amount of 3% w/v; and
a surfactant at an amount from 0.003 to 0.010 w/v.

2. The pharmaceutical composition of claim 1, wherein the mannitol is at a 5% w/v.

3. The pharmaceutical composition of any one of the preceding claims, wherein the surfactant is at an amount from 0.003 to 0.008, particularly in an amount of 0.004% w/v.

4. The pharmaceutical composition of any one of the preceding claims, wherein the buffering agent provides a pH 5 and it is selected from the group consisting of glycine, histidine, glutamate, succinate, phosphate, acetate, and aspartate; particularly, the buffering agent is histidine.

5. The pharmaceutical composition of any one of the preceding claims, wherein the surfactant is selected from the group consisting of sodium lauryl sulfate, dioctyl sodium sulfosuccinate, dioctyl sodium sulfonate, poloxamer, chenodeoxycholic acid, N-lauroylsarcosine sodium salt, lithium dodecyl sulfate, 1-octanesulfonic acid sodium salt, sodium cholate hydrate, sodium deoxycholate, glycodeoxycholic acid sodium salt, benzalkonium chloride or benzethonium chloride, cetylpyridinium chloride monohydrate, hexadecyltrimethylammonium bromide, 3-((3-cholamidopropyl)dimethylammonio)-1-propanesulfonate (CHAPS), 3-([3-Cholamidopropyl]dimethylammonio)-2-hydroxy-1-propanesulfonate (CHAPSO), SB3-10, SB3-12, digitonin, Triton X-100, Triton X-114, lauromacrogol 400, polyoxyl 40 stearate, polyoxyethylene hydrogenated castor oil 10, 40, 50 and 60, glycerol monostearate, polysorbate 20, 40, 60, 65 and 80, soy lecithin, Dipalmitoylphosphatidylcholine (DOPC), 1,2-Dimyristoyl-sn-glycero-3-phosphorylglycerol (DMPG), Dipalmitoylphosphatidylcholine (DMPC), and 18:1/18:1-Dioleoyl-phosphatidylgycerol (DOPG); sucrose fatty acid ester, methyl cellulose and carboxymethyl cellulose; particularly, the surfactant is polysorbate 20, polysorbate 80, and poloxamer 188; more particularly it is polysorbate 20.

6. The pharmaceutical composition of any one of the preceding claims, which comprises one of the following combinations of excipients:
I) histidine from 1 to 100 mM, pH 5; mannitol at 5% w/v; trehalose at 3% w/v; and polysorbate or poloxamer from 0.001 to 0.010% w/v;
II) histidine from 5 to 25 mM, pH 5; mannitol at 5% w/v; trehalose at 3% w/v; and polysorbate or poloxamer from 0.004 to 0.008% w/v;
III) histidine 10 mM, pH 5; mannitol at 5% w/v; trehalose at 3% w/v; and polysorbate or poloxamer at 0.004% w/v; or
IV) histidine 10 mM, pH 5; mannitol at 5% w/v; trehalose at 3% w/v; and polysorbate, particularly polysorbate 20, at 0.004% w/v.

7. The pharmaceutical composition of any one of the preceding claims, wherein the romiplostim is at a concentration between 0.25 and 250 mg/mL.

8. A pharmaceutical composition which is selected from the group consisting of the following compositions:
I) 0.5 mg/ml of romiplostim; histidine from 1 to 100 mM, pH 5; mannitol at 5% w/V; trehalose at 3% w/v; and polysorbate or poloxamer from 0.001 to 0.010% w/v;
II) 0.5 mg/ml of romiplostim; histidine from 5 to 25 mM, pH 5; mannitol at 5% w/v; trehalose at 3% w/v; and polysorbate or poloxamer from 0.004 to 0.008% w/v;
III) 0.5 mg/ml of romiplostim; histidine 10 mM, pH 5; mannitol at 5% w/v; trehalose at 3% w/v; and polysorbate or poloxamer at 0.004% w/v; and
IV) 0.5 mg/ml of romiplostim; histidine 10 mM, pH 5.0; mannitol 5% w/v; trehalose 3% w/v; and polysorbate, particularly polysorbate 20, 0.004% w/v.

9. The pharmaceutical composition of any one of the preceding claims, which is a lyophilized pharmaceutical composition.

10. A method for preparing a pharmaceutical composition as defined in any of the claims 1-8, comprising the step of mixing the excipients forming part of the combination as defined in any of the claims 1-8 with romiplostim.

11. A method for preparing a lyophilized pharmaceutical composition as defined in claim 9, the method comprising the steps of (i) preparing a solution comprising the combination of excipients as defined in any of the preceding claims 1-8 and the romiplostim; and (ii) lyophilizing the resulting solution.

12. A method for preparing a reconstituted pharmaceutical composition comprising the step of reconstituting the lyophilized composition as defined in claim 9.

13. A kit for preparing an aqueous pharmaceutical composition having a lyophilized pharmaceutical composition as defined in claim 9 and a second physiologically acceptable solvent for reconstituting the composition.

14. A pharmaceutical composition as defined in any one of the preceding claims 1-9 or the kit as defined in claim 13, for use in the treatment or prevention of thrombocytopenia; particularly of idiopathic or immune thrombocytopenia (ITP).
